(19) United States
(12) Patent Application Publication
(10) Pub. No.: US 2013/0019882 A1
(43) Pub. Date: Jan. 24, 2013

(54) Restraint for a bedfast patient

(71) Applicants: Danielle Durham, Columbia, SC (US); Amy Dixon Malcom, Columbia, SC (US)

(72) Inventors: Danielle Durham, Columbia, SC (US); Amy Dixon Malcom, Columbia, SC (US)

(21) Appl. No.: 13/135,904

(22) Filed: Jul. 18, 2011

(51) Int. Cl. A61F 5/37

(52) U.S. Cl. 128/876

(57) ABSTRACT

The restraint is disposed across the chest and under the armpits of a bedfast patient and includes a strap of elastic material, a layer of sheepskin material enveloping a central portion of the strap and a pair of ties, each of which is secured to and extends from a respective end of the elastic strap. The restraint keeps the patient in a fixed position and prevents the patient from sliding down in bed or leaning forward thus protecting indwelling medical devices from pulling and causing unwanted tension on the devices. The elasticity of the strap allows for adequate chest expansion during normal breathing and the sheepskin material helps to prevent skin breakdown from occurring across the chest and under the arms while absorbing moisture which helps to keep the skin of the patient dry.

This invention relates to a restraint used for a bedfast patient. More particularly, this invention relates to a restraint for keeping a patient in a hospital bed.

As is known, various types of restraint devices and systems have been provided for restraining various types of patients in a hospital bed. For example, some restraint types have been provided in the form of covers which envelope the entire body of the patient, for example, as described in U.S. Pat. Nos. 5,787,526 and 5,094,251. Another variation of this type of restraint system is described in U.S. Pat. No. 4,742,821 which employs a bag portion to restrain a patient.

Various types of restraining garments or vests have also been known to be fitted about a patient while seated and prior to laying the patient on a hospital bed. Examples of such types of garments are described in U.S. Pat. Nos. 4,119,095; 4,685,454; 4,777,944 and 7,319,400.

When a patient has multiple intravenous (IV) sites and tubing attached to medications on an infusion pump, in order to place a vest on an agitated patient, the medication must be stopped from running, the tubing from all the IV sites must be disconnected, and the vest placed on the patient while the patient may be thrashing about the bed. Then, all the tubing to the IV sites must be reconnected. This is because the arms must be placed through the vest.

Also, if a patient has a subclavian central line, the vest gets in the way of the tubing and rubs against the site and dressing. Access to the site is difficult as the vest is in the way and hinders access. Furthermore, the vest can compromise the integrity of the subclavian central line, potentially causing dislodgement of the line, unintentional dressing removal, and result in potential line site infections. This is mostly caused when the vest rubs against the site with patient movement.

Still other types of restraints systems have employed belts, for example as in U.S. Pat. No. 4,132,229 or net-like structures as described in U.S. Pat. No. 6,135,114.

The various types of restraints provide different advantages but also have their limitations. For example, the restraints that rely upon garments or vests require the patient to be in a seated or raised position in order to clothe the patient with the garment prior to being laid onto a hospital bed. This is difficult for an agitated uncooperative patient. Restraints of the strap type can be tightened to such a degree as to render the patient uncomfortable when lying in a supine position or to prevent a patient from being able to breathe properly or make limited motions in a bed. The vest does not stop a patient from sliding down in bed. This has been known to cause skin breakdown under the arm and tracheotomy irritation because the vest stays in place, being secured to a fixed point on the bed, and the patient does not.

Accordingly, it is an object of the invention to provide a restraint for a bedfast patient that can be easily used to restrain an agitated patient and to prohibit medical devices from becoming compromised when an agitated patient is thrashing about in bed.

It is another object of the invention to provide a restraint that allows a bedfast patient to breathe properly allowing for normal breathing pattern and adequate chest expansion.

It is another object of the invention to provide a restraint that does not interfere with indwelling medical devices during placement and duration of restraint use.

It is another object of the invention to provide a restraint that can be readily applied to and removed from an agitated patient by a single attendant.

Briefly, the invention provides a restraint that is comprised of an elongated strap of webbed elastic material, a layer of sheepskin material enveloping a central portion of the strap and a pair of ties, each of which is secured to and which extends from a respective end of the strap.

The strap of webbed elastic material is made of sufficient strength to restrain a patient in a supine position from leaning forward potentially putting tension on IV tubing or other important medical devices. For example, the strap is made of a combination of webbed spandex, polyester and/or nylon materials. In addition, the strap is free of latex.

The width of the strap may be from 3 inches to 6 inches depending on the size of a patient. Typically, the preferred width is 4 inches. The length of the restraint is from 3 to 3.5 feet.

The strap is characterized in being elastically stretchable longitudinally to a sufficient degree to allow a patient to breathe in a normal pattern when the strap is firmly secured across the chest. That is to say, the elasticity of the strap allows for adequate chest expansion when the strap is firmly secured across the chest of the patient. The webbed elastic material is strong enough to prevent a patient from leaning forward but will not hinder normal breathing patterns.

The layer of sheepskin material may be made of a single strip of material which is wrapped about the elastic strap and secured in place by any suitable means, for example by sewing, gluing, snap fasteners and the like. As is known, sheepskin contains lanolin which is a natural antimicrobial, antibacterial and antifungal agent. In addition, sheepskin is non-abrasive to the skin. The sheepskin helps to prevent friction and/or shear, thus keeping skin breakdown from occurring across the patient chest and under the arms of the patient. The sheepskin material readily absorbs moisture which keeps the skin dry thus preventing further skin breakdown.

Typically, the restraint device is placed across the chest and under the arm pits of a patient lying in a bed. The restraint is such as to prevent the patient from leaning forward or sliding down in the bed so that the patient cannot reach indwelling medical devices with their hands when restrained properly. The elasticity of the strap provides for a snug firm fit across the chest while also allowing for adequate chest expansion. The elasticity of the strap also prevents a patient from leaning too far forward and potentially pulling at indwelling medical devices with their hands when used in conjunction with hospital-approved wrist restraints.

The ties which are attached to the end of the restraint are durable medical-grade ties that are sewn to each end of the strap. These ties allow for a secure fastening of the restraint to a bed frame. A slip knot is used to secure the ties in place as a slip knot will allow a quick release of the restraint from a patient in case of an emergency.

These and other objects and advantages of the invention will become more apparent from the following detailed description taken in conjunction with the accompanying drawings wherein;

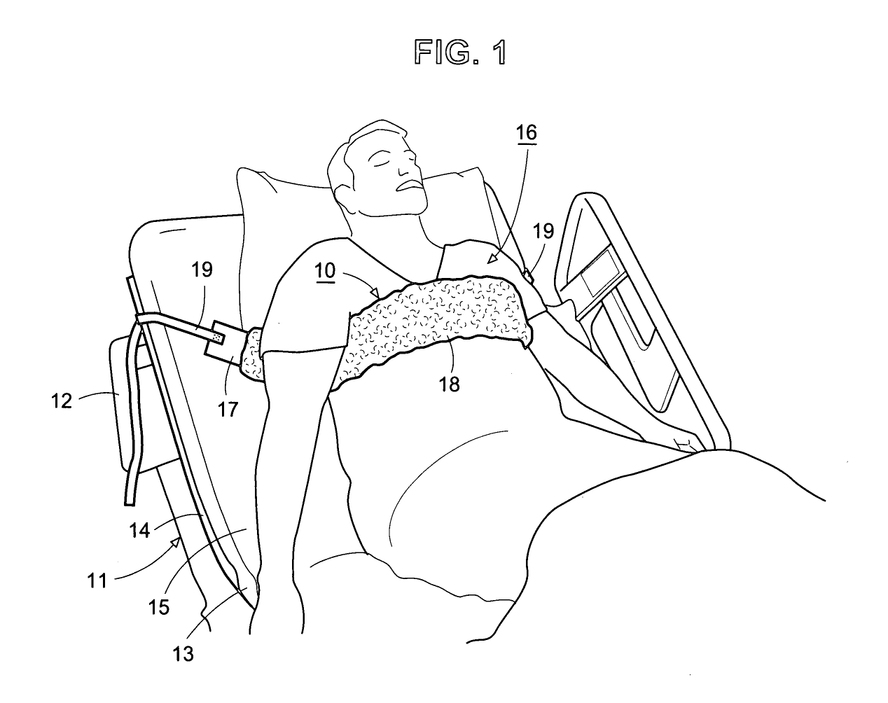

FIG. 1 illustrates a schematic view of a patient in a hospital bed with a restraint in place in accordance with the invention.

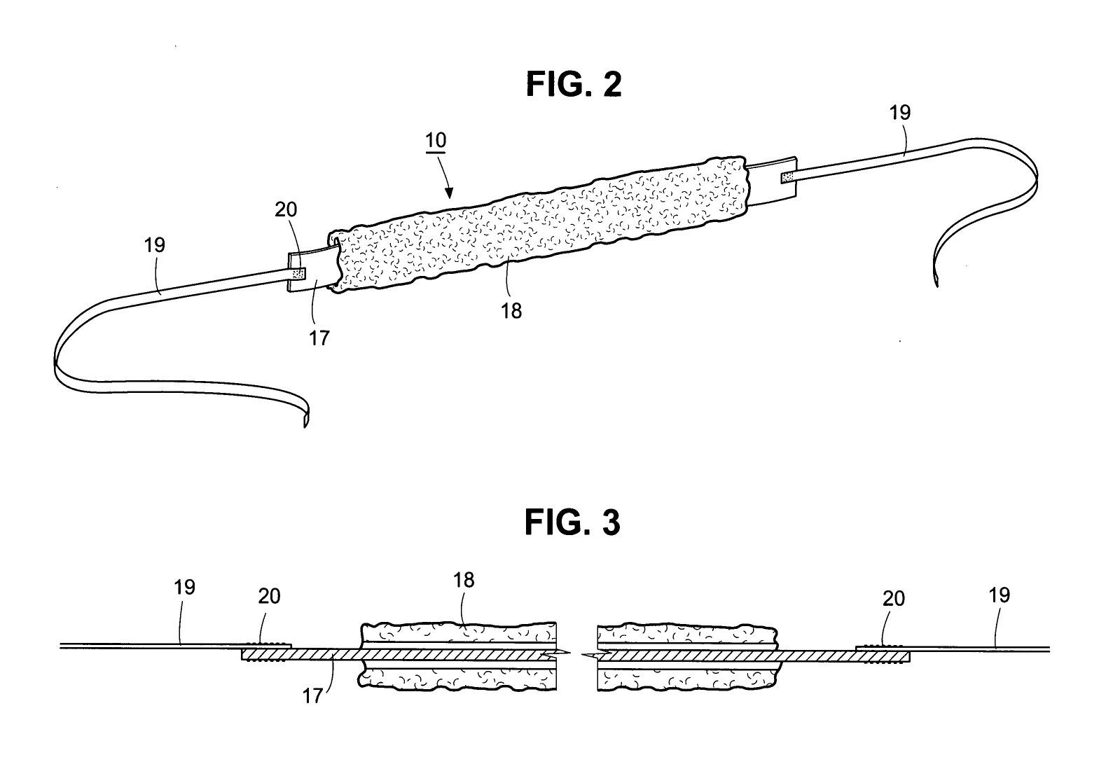

FIG. 2 illustrates a view of the restraint in accordance with the invention;

FIG. 3 illustrates a cross sectional view of the restraint taken on line 3-3 of FIG. 2;

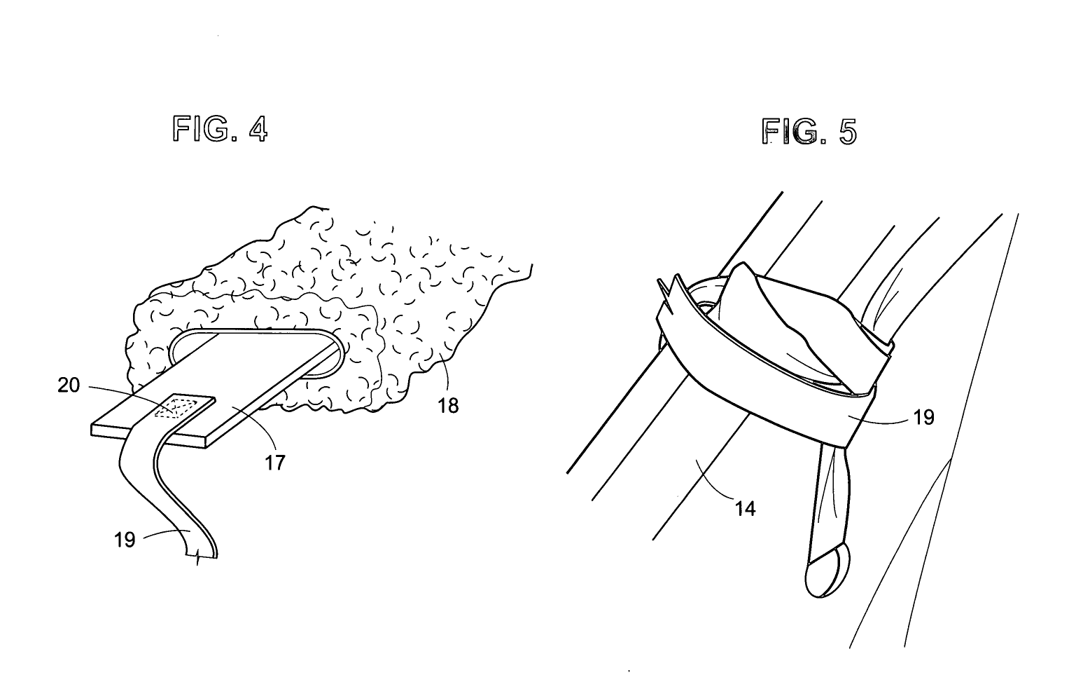

FIG. 4 illustrates a partial view of the securement of a tie to the strap of the restraint in accordance with the invention;

FIG. 5 illustrates a manner of securing a tie to a hospital bed.

Referring to FIG. 1, the restraint 10 is used in combination with a conventional hospital bed 11 having a frame 12 with a fixed lower section 13 and a movable upper portion 14 and a mattress 15 on the frame 12. In the illustrated embodiment, the upper portion 14 is movable relative to the lower section 13 between a lowered position (not shown) with a patient 16 in a supine position and a raised position, as shown, with the patient 16 in a raised position.

Referring to FIGS. 2 and 3, the restraint 10 includes an elongated strap 17, a layer of sheepskin material 18 enveloping a central portion of the strap 17 and pair of ties 19, each of which is secured to a respective end of the strap 17.

The elongated strap 17 is made of elastic material which is characterized in being elastically stretchable longitudinally thereof. For example, the elastic material is latex-free made of any suitable material, such as a combination of spandex, polyester and/or nylon.

The elasticity of the strap is such as to provide a snug firm fit around the chest of a patient 16 while also allowing for adequate chest expansion during normal breathing; additionally, the strap is strong enough to hold the patient securely in bed and heavily restrict the patient from sitting up or sliding down in bed. The elasticity of the material is sufficient to keep the patient 18 from leaning too far forward and potentially pulling out medical indwelling devices when the patient's hands are used in conjunction with hospital-approved wrist restraints. Depending on the patient, the strap 17 may be made of a wider width for large patients and a narrower width for young children and may be made with different degrees of stretchability for such patients.

The width of the strap 17 is sufficient to apply a restraining force onto the chest of the patient 16 in a comfortable manner which does not impose concentrated forces on the patient 16.

The strap 17 is preferably made as a single web of material which is folded on itself to form a two- or three-ply layer for increased strength and durability.

The layer of sheepskin material 18 envelopes a central portion of the strap 17 and is secured thereon in a suitable manner. For example, the sheepskin material 18 may be made of a single web that is wrapped circumferentially about the strap 17 and secured along the two longitudinal edges by suitable means, for example by sewing, gluing, snap-fasteners, zipper and the like to form a longitudinal seam. The securement is such that a longitudinal seam does not become exposed as this may otherwise chaff against the skin of a patient and cause skin breakdown.

The securement of the sheepskin material 18 to the strap 17 may be of a permanent nature. Alternatively, the sheepskin material 18 may be secured to the strap 17 in a removable manner so that the sheepskin material 18 may be removed from the strap 17 and the two elements cleaned and sterilized in a separate manner.

Referring to FIGS. 3 and 4, each tie 19 is secured to the end of the strap 17 by any suitable fastening means, such as by stitching 20. Each tie 19 is made of a suitable material and is of a narrower width than the strap 17. For example, each tie 19 has a width of one inch.

As shown in FIG. 5, each tie 19 is sufficiently pliable so as to be tied to the movable upper section 14 of the frame 12 of the bed 10, for example using a slip knot. Thus, when the upper section 14 of the frame 12 is moved between a raised position, as shown in FIG. 1, and a lowered position with the patient 16 in a supine position, the restraint 10 moves with the upper section 14 of the frame 12. The ability to quickly remove the restraint 10 from the patient 16 in the case of an emergency is of particular advantage.

Referring to FIG. 1, when the restraint 10 is placed in use, the layer of sheepskin material 18 is laid across the patient's chest and under the patient's armpits. The sheepskin material 18 helps to prevent friction and/or shear thus keeping skin breakdown from occurring across the chest and under the arms. Further, the sheepskin material 18 readily absorbs moisture which helps to keep the skin of the patient dry. This is important for hyperthermic patients. Use of the sheepskin material 18 prevents skin breakdown.

Proper placement of the restraint 10 across the chest and under the armpits of the patient 18 does not obstruct or tamper with indwelling medical devices that may be attached to the patient 18 and allows for tracheostomy/airway protection, i.e., the restraint 10 will not come into contact with artificial airways. The position of the restraint 10 allows a subclavian line site, IV sites, chest tube sites, and the like, to be easily seen and accessible.

Because the restraint 10 is secured under the armpits of the patient 16, the restraint 10 will not be able to rub against a tracheostomy site, subclavian central line site, and the like, even if the patient were to slide down in bed or sit up slightly. A vest restraint is basically an inch or two from the trachostomy site when properly applied, not taking into account when a patient slides or sits up, which could be uncomfortable, rub against, irritate, or become detrimental to any artificial airway. Also, when a patient is wearing a vest properly, a patient has to ability to slide down in bed. Thus, should the patient slide down in the bed, the vest does not because the vest is secured to a fixed position. As a result, the vest stays in place and the patient does not, thus, having the patient appear to be “hanging” themselves with the vest.

The way the restraint 10 is constructed and located under the armpits, the patient is prevented from sliding down or leaning forward in bed when the patient's hands are secured with hospital approved wrist restraints.

The restraint 10 may be easily and quickly applied to an agitated, combative, confused patient 16 and removed with only one person required during emergent conditions. In emergent conditions, a vest needs to be cut off.

The invention thus provides a restraint 10 that prevents a patient from sitting up in bed so that indwelling medical devices such as ET tubes, IV lines, nasogastric tubes, chest tubes and the like are protected when the wrist restraints are being used. When a patient can lean forward in bed, wearing a vest or not, this puts tension on medical devices potentially causing harm to the patient and patient medical devices.

The compact design of the restraint 10 allows for improved patient comfort as less material is required to make the restraint 10 and is ideal for patients with elevated temperatures or moist skin.

The restraint 10 provides less constricting material, when compared to the vest, decreased agitation and anxiety in confused patients.

In addition, the restraint 10 is aesthetically pleasing, the sheepskin material 18 is soft and comfortable and the elastic strap 17 is non-constricting. With this restraint 10, patients do not feel as “tied down” and “restrained” as with the vest restraints.

The restraint 10 serves to protect indwelling medical devices by keeping the patient in a fixed position. For example, when a patient has numerous IV sites with tubing attached, the restraint 10 does not interfere with these sites and no intervention, such as, unhooking tubing from IV sites, is needed to place the restraint 10 about the patient.

What is claimed is:

1. A restraint comprising
an elongated strap of elastic material characterized in being elastically stretchable longitudinally thereof;
a layer of medical grade sheepskin material enveloping a central portion of said strap; and
a pair of ties, each said tie being secured to and extending from a respective end of said strap.

2. A restraint as set forth in claim 1 wherein said elongated strap is webbed and made of latex-free material.

3. A restraint as set forth in claim 1 wherein said elongated strap has a width of from 4 to 6 inches.

4. A restraint as set forth in claim 1 wherein each said tie is of medical grade.

5. In combination,
a hospital bed having a frame and a patient receiving portion within said frame; and
a restraint for maintaining a patient on said patient receiving portion in a supine position, said restraint including an elongated strap, a layer of sheepskin material enveloping a central portion of said strap for disposition across an upper portion of a patient received on said patient receiving portion of said bed, and a pair of ties, each said tie being secured to and between a respective end of said strap and said patient receiving portion of said bed.

6. The combination as set forth in claim 5 wherein said patient receiving portion of said bed has an upper section, said upper portion being movable relative to said frame between a lowered position with a patient in a supine position thereon and a raised position with the patient in a raised position thereon.

7. The combination as set forth in claim 6 wherein said strap is of a material characterized in being elastically stretchable longitudinally thereof to a sufficient degree to allow a patient on said patient receiving portion to breathe in a normal pattern.
